SEQUENCE LISTING (11 sequences)

SEQ ID NO: 1 - DNA, 1428 bp, Ralstonia eutropha
ttgttttccgcagcaacagatgtactttaaggtatatgctgcagagcaagatcgttagcacggtcgttcggttaaaggcataatcggcgccacgatcccccggtgtattgtcagtacattagtacttggtagtacgtacctcttagctagacccaggcagaaaaggcatgctctaccaattgcatgagttccagcgctcgatcctgcacccgctgaccgcgtgggcccaggcgaccgccaagaccttcaccaaccccctcagcccgctctcgctggttcccggcgcaccccgcctggctgccggctatgaactgctgtaccggctcggcaaggaatacgaaaagccggcattcgacatcaagtcggtgcgctccaacgggcgcgacatccccatcgtcgagcagaccgtgcttgaaaagccgttctgcaagctggtgcgcttcaagcgctatgccgacgacccggagaccatcaagctgctcaaggatgagccggtggtgctggtggccgcgccgctgtcgggccaccatgccacgctgctgcgcgacacggtgcgcacgctgctgcaggaccacaaggtctacgtcaccgactggatcgacgcacgcatggtgccggtcgaggaaggcgcgttccacctgtcggactacatctactacatccaggaattcatccgccatatcggcgccgagaacctgcatgtgatctcggtatgccagcccaccgtgccggtgctggccgcgatctcgctgatggcctcggccggcgagaagacgccgcgcaccatgaccatgatgggcggcccgatcgacgcccgcaagagccccaccgcggtcaactcgctggcgaccaacaagtcgttcgagtggttcgagaacaacgtcatctacaccgtgccggccaactaccccggccacggccgccgcgtctacccgggctttttgcagcatgccggtttcgtggcgatgaacccggaccggcacctttcctcgcactatgacttctacctgagcctggtcgagggcgatgcggatgacgccgaagcccacgtgcgcttctacgacgaatacaacgcggtgctcgacatggccgccgagtactacctcgacaccatccgcgaggtgttccaggaattccgcctggccaacggcacctgggccatcgacggcaatccggtgcggccgcaggacatcaagagcaccgcgctgatgaccgtcgagggcgaactggacgacatctcgggcgcgggccagaccgccgcggcgcacgacctgtgcgccggcatcccgaaaatccgcaagcagcacctgaacgcggcacactgcggccactacggcatcttctcgggccggcgctggcgcgaagagatctacccgcagctgcgcgactttatccgcaagtaccaccaggcctcggccaccaggtaag

SEQ ID NO: 2 - DNA, 580 bp, Escherichia coli
aacaaactccgggaggcagcgtgatgcggcaacaatcacacggatttcccgtgaacggtctgaatgagcggattattttcagggaaagtgagtgtggtcagcgtgcaggtatatgggctatgatgtgcccggcgcttgaggctttctgcctcatgacgtgaaggtggtttgttgccgtgttgtgtggcagaaagaagatagccccgtagtaagttaattttcattaaccaccacgaggcatccctatgtctagtccacatcaggatagcctcttaccgcgctttgcgcaaggagaagaaggccatgaaactaccacgaagttcccttgtctggtgtgtgttgatcgtgtgtctcacactgttgatattcacttatctgacacgaaaatcgctgtgcgagattcgttacagagacggacacagggaggtggcggctttcatggcttacgaatccggtaagtagcaacctggaggcgggcgcaggcccgccttttcaggactgatgctggtctgactactgaagcgcctttataaaggggctgctggttcgccggtagcccctttctccttgctgatgttgt

SEQ ID NO: 3 - DNA, 4193 bp, Ralstonia eutropha
ttgacagcgcgtgcgttgcaaggcaacaatggactcaaatgtctcggaatcgctgacgattcccaggtttctccggcaagcatagcgcatggcgtctccatgcgagaatgtcgcgcttgccggataaaaggggagccgctatcggaatggacgcaagccacggccgcagcaggtgcggtcgagggcttccagccagttccagggcagatgtgccggcagaccctcccgctttgggggaggcgcaagccgggtccattcggatagcatctccccatgcaaagtgccggccagggcaatgcccggagccggttcgaatagtgacggcagagagacaatcaaatcatggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatgacgcttgcatgagtgccggcgtgcgtcatgcacggcgccggcaggcctgcaggttccctcccgtttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggggttttccggggccgcgcgcggttggcgcggacccggcgacgataacgaagccaatcaaggagtggacatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 4 - DNA, 35 bp, Artificial Sequence
  Synthetic Construct
gctctagaggatccttgttttccgcagcaacagct

SEQ ID NO: 5 - DNA, 30 bp, Artificial Sequence
  Synthetic Construct
cgggatccaagcttacctggtggccgaggc

SEQ ID NO: 6 - DNA, 34 bp, Artificial Sequence
  Synthetic Construct
gcgaattctttaaagacgcgcgcatttctaaact

SEQ ID NO: 7 - DNA, 33 bp, Artificial Sequence
  Synthetic Construct
gcggtaccgagctccgggttgatcgcacagtca

SEQ ID NO: 8 - DNA, 35 bp, Artificial Sequence
  Synthetic Construct
ggcgagctcgcgcatgcccgaccgctgaacagctg

SEQ ID NO: 9 - DNA, 35 bp, Artificial Sequence
  Synthetic Construct
gcaagctttttaaagcgcagttaagcaagataatc

SEQ ID NO: 10 - DNA, 35 bp, Artificial Sequence
  Synthetic Construct
gctctagagagctcaaagccacgttgtgtctcaaa

SEQ ID NO: 11 - DNA, 30 bp, Artificial Sequence
  Synthetic Construct
gcgcatgcttagaaaaactcatcgagcatc

(19) United States
(12) Patent Application Publication
(10) Pub. No.: US 2005/0069995 A1
(43) Pub. Date: Mar. 31, 2005

(54) (R)-hydroxycarboxylic acid producing recombinant microorganism and process for preparing (r)-hydroxycarboxylic acid using the same

(76) Inventors: Sang-Yup Lee, Taejon (KR); Young Lee, Taejon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/496,543

(22) PCT Filed: Nov. 29, 2001
(86) PCT No.: PCT/KR01/02060

(30) Foreign Application Priority Data
Nov. 28, 2001 (KR) 2001-74676

(51) Int. Cl. C12P 7/42; C12N 1/21

(52) U.S. Cl. 435/146; 435/252.33

(57) ABSTRACT

The present invention relates to recombinant microorganisms carrying both a gene encoding intracellular polyhydroxyalkanoate (PHA) depolymerase and a gene encoding PHA biosynthesis-related enzymes, and a process for preparing optically pure (R)-hydroxycarboxylic acids by culturing the recombinant microorganisms. The present invention provides: a recombinant microorganism transformed with two recombinant plasmids each of which contains a gene encoding intracellular PHA depolymerase and a gene encoding PHA biosynthesis-related enzymes, respectively; a recombinant microorganism harboring a gene encoding PHA biosynthesis-related enzymes in an integrated form with its chromosome, which is transformed with a recombinant plasmid containing a gene encoding intracellular PHA depolymerase; and, a process for preparing optically pure (R)-hydroxycarboxylic acids by culturing the recombinant microorganisms. In accordance with the present invention, (R)-hydroxycarboxylic acids can be released from the recombinant microorganisms into culture media after depolymerizing most PHA produced from the said microorganisms, which makes possible practical preparation of (R)-hydroxycarboxylic acids in a simple manner with reduced waste of substrates to increase the productivity, finally to allow the mass production of various optically pure (R)-hydroxycarboxylic acids.

### BACKGROUND OF THE INVENTION

1. Field of the Invention

The present invention relates to recombinant microorganisms producing (R)-hydroxycarboxylic acids and a process for preparing (R)-hydroxycarboxylic acids by employing the same, more specifically, to recombinant microorganisms carrying both a gene encoding intracellular PHA depolymerase and a gene encoding polyhydroxyalkanoate (PHA) biosynthesis-related enzymes, and a process for preparing optically pure (R)-hydroxycarboxylic acids by culturing the said recombinant microorganisms where biosynthesis and degradation of PHA occur simultaneously.

2. Description of the Prior Art

Since (R)-hydroxycarboxylic acid carries two functional groups, i.e., hydroxyl group (—OH) and carboxyl group (—COOH), it is readily employed for the organic syntheses of a variety of useful materials while providing chiral centers to the materials. Furthermore, the two functional groups can be converted readily into other forms, which makes possible its wide use of a chiral precursor compound in fine chemical fields. In addition, (R)-hydroxycarboxylic acid may be used as a promising precursor for the syntheses of antibiotics, vitamins, aromatics and pheromones, nonpeptide ligand for the design of medical and pharmaceutical products, and a precursor for novel pharmaceuticals, especially of carbapenem antibiotics which draw attentions as an alternative of penicillin (see: Lee et al., Biotechnol. Bioeng., 65:363-368, 1999). For example, it has been reported that the method for synthesis of (+)-thiennamycin from methyl-(R)-3-hydroxybutyrate was developed (see: Chiba and Nakai, Chem. Lett., 651-654, 1985).

On the other hand, polyhydroxyalkanoate (PHA), a polyester formed by ester linkage of hydroxycarboxylic acids, is an energy and carbon source which is synthesized and accumulated in many species of microorganisms. Due to the optical specificity of its biosynthetic enzyme, hydroxycarboxylic acid as a monomer of PHA has only (R)-type optical activity, except for the compounds such as 4-hydroxybutyric acid which has no optical isomers. Accordingly, optically pure (R)-3-hydroxycarboxylic acids can be produced simply by depolymerizing the biosynthesized PHA. On the ather hand, chemical processes for preparing (R)-3-hydroxybutyrate, alkyl-(R)-3-hydroxybutyrate, or alkyl-(R)-3-hydroxyvalerate via depolymerization of poly-(hydroxybutyrate) (PHB) and poly(3-hydroxybutyrate-co-3-hydroxyvalerate) (PHB/V) have been reported in the art (see: Seebach et al., Org. Synth., 71:39-47, 1992; Seebach and Zuger, Helvetica Chim. Acta, 65:495-503, 1982). However, the said processes for preparing (R)-3-hydroxybutyrate via chemical means have revealed several disadvantages of low yield due to complicated steps of culture of microorganism, recovery of cells, isolation of polymers, depolymerization and isolation/purification, and consumption of a large quantity of organic solvents. Moreover, a great deal of microbial cell mass are produced as a byproduct, which naturally limits trial of producing (R)-hydrocarboxylic acids merely to (R)-3-hydroxybutyrate and (R)-3-hydroxyvalerate.

Recently, the present inventors have reported a process for preparing various (R)-3-hydroxycarboxylic acids including (R)-3-hydroxybutyrate via autodegradation (depolymerization) employing PHA depolymerase, which occurs naturally with PHA biosynthesis-related enzymes in the PHA-producing microorganisms (see: Lee et al., Biotechnol. Bioeng., 65:363-368, 1999). The autodegradation method is more efficient than conventional chemical methods, for example, after overproducing PHB in Alcaligenes latus by fermentation, incubation for 30 min under a proper pH condition would allow the microorganism to degrade PHB into (R)-hydroxybutyrate with over 95% optical purity, which is then released into a medium. Although the autodegradation method can be applied to produce various (R)-3-hydroxycarboxylic acids, it essentially accompanies a batch process in which PHA is accumulated and then degraded. If biosynthesis and degradation of PHA could occur simultaneously in a continuous manner, it could be expected that by-products of microbial materials can be dramatically reduced finally to increase the yield of hydoxycarboxylic acids from the substrate. In this regard, there has been a continuing need to develop a process for preparing (R)-3-hydroxycarboxylic acids by a simple continuous process to produce (R)-3-hydroxycarboxylic acids in a more efficient and economical way.

The general mechanism of biosynthesis and depolymerization of PHA in a microorganism is as follows: When a microorganism is subject to an unbalanced growth condition of sufficient carbon source and limited essential elements such as nitrogen, phosphate, or magnesium, it turns on the expression of the enzymes for PHA biosynthesis and then synthesizes and accumulates PHA inside the cell body from excessive carbon source through PHA biosynthesis (see: Lee, Biotechnol. Bioeng., 49:1-14, 1996). Later, when supply of essential elements are resumed, PHA is degraded into its monomer, (R)-3-hydroxycarboxylic acid, by the action of PHA depolymerase and oligomeric hydrolysis enzymes (see: Muller and Seebach, Angew. Chem. Int. Ed. Engl., 32:477-502, 1993).

The mechanism for recycling of (R)-3-hydroxycarboxylic acid in the metabolic pathway whithin a microorganism has been established only for (R)-3-hydroxybutyrate as follows: By the action of (R)-3-hydroxybutyrate dehydrogenase, (R)-3-hydroxybutyrate is converted into acetoacetate which is then recycled in the metabolic pathway of a microorganism (see: Muller and Seebach, Angew. Chem. Int. Ed. Engl., 32:477-502, 1993; Lee et al., Biotechnol. Bioeng., 65:363-368, 1999). Accordingly, both PHA biosynthesis-related enzymes and PHA depolymerase are required for the production of (R)-3-hydroxycarboxylic acids in the microorganism, and for the production of (R)-3-hydroxybutyrate, it is preferable to inhibit or remove (R)-3-hydroxybutyrate dehydrogenase activity.

The present inventors and many other researchers have conducted studies on the development of an efficient process for PHA production employing recombinant E. coli, and in case of PHB or PHB/V, so favorable technical progress has been made to a level that products can be accumulated up to over 80% of total dried cell mass (see: Slater et al., J. Bacteriol., 170:4431-4436, 1988; Schubert et al., 170, 5837-5847, 1988; Kim et al., Biotechnol. Lett., 14:811-816, 1992; Fidler and Dennis, FEMS Microbiol. Rev., 103:231-236, 1992; Lee et al., J. Biotechnol., 32:203-211, 1994; Lee et al., Ann. NY Acad. Sci., 721:43-53, 1994; Lee et al., Biotechnol. Bioeng., 44:1337-1347, 1994; Lee and Chang, J. Environ. Polymer Degrad., 2:169-176, 1994; Lee and Chang, Can. J. Microbiol., 41:207-215, 1995; Yim et al., Biotechnol. Bioeng., 49:495-503, 1996; Lee and Lee, J. Environ. Polymer Degrad., 4:131-134, 1996; Wang and Lee, Appl. Environ. Microbiol., 63:4765-4769, 1997; Wang and Lee, Biotechnol. Bioeng., 58:325-328, 1998; Lee, Bioprocess Eng., 18:397-399, 1998; Choi et al., Appl. Environ. Microbiol, 64:4897-4903, 1998; Wong and Lee, Appl. Microbial. Biotechnol., 50:30-33, 1998; and, Lee et al., Int. J. Biol. Macromol., 25:31-36, 1999).

In nature, E. coli neither synthesize PHA as an intracellular storage material for energy nor have PHA depolymerase, and it is not considered to have (R)-3-hydroxybutyrate dehydrogenase which converts (R)-3-hydroxybutyrate into acetoacetate. PHA-synthesizing recombinant E. coli, which is constructed by introducing genes encoding PHA synthesis-related enzymes from other species, does not degrade the resulting PHA produced and accumulated in its cellular space because the recombinant E. coli carries only PHA biosynthesis-related enzymes (see: Lee, Trends Biotechnol., 14:98-105, 1996; Lee, Nature Biotechnol., 15:17-18, 1997). Therefore, the present inventors have perceived that (R)-3-hydroxycarboxylic acid, especially (R)-3-hydroxybutyrate can be produced more efficiently by co-introducing/co-expressing the genes for PHA-synthesizing enzymes and PHA depolymerase in a recombinant E. coli, and furthermore, (R)-3-hydroxybutyrate would not be metabolized to acetoacetate in the absence of (R)-3-hydroxybutyrate dehydrogenase. This perception had inspired the present inventors to develop a recombinant microorganism co-transformed with genes for both PHA biosynthesis-related enzymes and intracellular PHA depolymerase, and to develop a process for preparing (R)-hydroxycarboxylic acids by employing the same (see: Korean Patent Appln. No. 10-2000-0026158). However, the process for preparing (R)-hydroxycarboxylic acids provided by the present inventors has several disadvantages that it may increase the unit cost of production due to low productivity and a waste of substrates resulting from the presence of undegradated PHA within the microorganism after the culture.

Under the circumstances, there are strong reasons for developing a process for preparing optically active (R)-3-hydroxycarboxylic acids by depolymerizing produced PHA into (R)-3-hydroxycarboxylic acids in a more efficient manner while reducing the waste of substrates, finally to increase the productivity of (R)-3-hydroxycarboxylic acids.

### SUMMARY OF THE INVENTION

The present inventors have made an effort to develop an efficient process for preparing optically active (R)-3-hydroxycarboxylic acids with increased productivity, and found that various optically pure (R)-hydroxycarboxylic acids can be prepared in a massive manner, by culturing microorganisms carrying both a gene encoding intracellular PHA depolymerase and a gene encoding PHA biosynthesis-related enzymes, where biosynthesis and degradation of PHA occur simultaneously and the depolymerization of almost all of the prepared PHA into (R)-hydroxycarboxylic acids is accomplished.

A primary object of the present invention is, therefore, to provide recombinant microorganisms producing optically pure (R)-hydroxycarboxylic acids.

The other object of the invention is to provide a process for preparing optically pure (R)-3-hydroxycarboxylic acids by culturing the said recombinant microorganisms.

### BRIEF DESCRIPTION OF THE DRAWINGS

The above and the other objects and features of the present invention will become apparent from the following descriptions given in conjunction with the accompanying drawings, in which:

FIG. 1 is a genetic map of a recombinant plasmid of the invention, pUC19Red.

FIG. 2 is a genetic map of a recombinant plasmid of the invention, pUC19Red_stb.

FIG. 3 is a genetic map of a recombinant plasmid of the invention, p5184.

FIG. 4 is a graph showing the concentrations of dried-cell, polyhydroxybutyrate (PHB) and (R)-hydroxybutyrate monomer before and after pyrolysis under a basic condition, the rates of PHB degradation and PHB biosynthesis, and the ratio of PHB degradation rate/PHB biosynthesis rate, depending on the elapsed time during the culture of a recombinant E. coli of the invention, XL1-Blue/pSYL105Red.

FIG. 5 is a graph showing the concentrations of dried-cell, PHB and (R)-hydroxybutyrate monomer before and after pyrolysis under a basic condition, the rates of PHB degradation and PHB biosynthesis, and the ratio of PHB degradation rate/PHB biosynthesis rate, depending on the elapsed time during the flask culture of a recombinant E. coli of the invention, XL1-Blue/pUC19Red;p5184.

FIG. 6 is a graph showing the concentrations of dried-cell, PHB and (R)-hydroxybutyrate monomer concentration before and after pyrolysis under a basic condition, and pH, depending on the elapsed time during the batch culture of a recombinant E. coli of the invention, B-PHA+/pUC19Red_stb.

### DETAILED DESCRIPTION OF THE INVENTION

The present invention provides: a recombinant microorganism transformed with two recombinant plasmids each of which contains a gene encoding intracellular PHA depolymerase (SEQ ID No: 1) and a gene encoding PHA biosynthesis-related enzymes (SEQ ID No: 3), respectively; a recombinant microorganism containing a gene encoding PHA biosynthesis-related enzymes in an integrated form with its chromosome, which is transformed with a recombinant plasmid containing a gene encoding intracellular PHA depolymerase; and, a process for preparing optically pure (R)-hydroxycarboxylic acids by culturing the recombinant microorganisms. The recombinant microorganism which is co-transformed with both a gene encoding intracellular PHA depolymerase and a gene encoding PHA biosynthesis-related enzymes, carries a higher copy number of the gene encoding intracellular PHA depolymerase than that of PHA biosynthesis-related enzymes. Although it is preferable that the said genes originate from Ralstonia eutropha, those of other species may be employed in the invention. The gene encoding PHA depolymerase is present in a plasmid in an inserted form, which may further contain parB(hok/sok) locus originating from R1, E. coli plasmid. The gene encoding PHA biosynthesis-related enzymes may be present within a locus for phosphotransacetylase (Pta) in an integrated form with its chromosome. A microorganism for transformation includes E. coli, preferably, E. coli XL1-Blue or E. coli B. Culture of the recombinant microorganism can be carried out by a continuous or batch process, where (R)-hydroxycarboxylic acids produced therefrom include (R)-3-hydroxybutyrate and (R)-3-hydroxyvalerate in a monomeric or a dimeric form.

The present invention is further illustrated in more detail as follows.

Using two plasmid vectors which can coexist in a microorganism with a different copy number, the present inventors introduced a gene encoding PHA biosynthesis-related enzymes into a plasmid of low copy number, and introduced a gene encoding intracellular PHA depolymerase into a plasmid of high copy number, with an intention that the recombinant microorganisms transformed with the plasmids express a larger amount of intracellular PHA depolymerase than PHA biosynthesis-related enzymes, to produce and release hydroxycarboxylic acids including (R)-3-hydroxybutyrate without intracellular accumulation of PHA. Furthermore, based on the unexpected finding that the productivity and yield of (R)-hydroxycarboxylic acid was rapidly increased rather than decreased when the expression level of PHA biosynthesis-related enzymes was decreased, they tried to produce hydroxycarboxylic acids including (R)-3-hydroxybutyrate efficiently and release into liquid media, by culturing a recombinant E. coli transformed with a plasmid of high copy number, which contains a gene encoding intracellular PHA depolymerase, while containing a gene encoding PHA biosynthesis-related enzymes in an integrated form with its chromosome.

After all, the process of the present invention comprises a step of preparing optically pure (R)-hydroxycarboxylic acid released into the liquid media, by culturing a recombinant microorganism transformed with both a recombinant plasmid of high copy number expressing intracellular PHA depolymerase and a recombinant plasmid of relatively low copy number expressing PHA biosynthesis-related enzymes, or, a recombinant microorganism transformed with a plasmid of high copy number expressing intracellular PHA depolymerase while containing a gene encoding PHA biosynthesis-related enzymes in an integrated form with its chromosome.

In accordance with the present invention, the recombinant microorganism transformed with both a gene encoding intracellular PHA depolymerase and a gene encoding PHA biosynthesis-related enzymes, is cultured using glucose as a substrate, and produces monomers and dimers of (R)-3-hydroxybutyrate, which are then released into liquid media upon the expression of the said enzymes. (R)-3-hydroxybutyrate and its dimers thus released can be separated on LC or HPLC under a specified analysis condition. If required, the dimer can be degraded into (R)-3-hydroxybutyrate by way of heating it under a basic condition (see: Lee et al., Biotechnol. Bioeng., 65:363-368, 1999).

The present inventors isolated a DNA fragment (SEQ. ID No: 1) including a gene encoding intracellular PHA depolymerase and its ribosome-binding site by means of polymerase chain reaction (PCR) with reference to DNA sequence from Ralstonia eutropha accessible on GenBank (see: Saito and Saegusa, GenBank Sequence Database, AB017612, 2001; Saegusa et al., J. Bacteriol., 183:94-100, 2001), and then introduced the said fragment into pUC19 (New England Biolabs, USA) of high copy number, finally to prepare pUC19Red. Further, for the purpose of increasing the stability of a plasmid, pUC19Red was introduced by a DNA fragment containing parB(hok/sok) locus (SEQ. ID No. 2) (see: GenBank Sequence Data, X05813; Gedes, Bio/Technology, 6:1402-1405, 1988) to give pUC19Red-stb, where the DNA fragment was obtained by the digestion of pSYL105(see: Lee et al., Biotechnol. Bioeng., 44: 1337-1347, 1994) with the restriction endonuclease EcoRI and BamHI.

Meanwhile, a DNA fragment (SEQ. ID No: 3) containing a gene for operon which comprises PHA biosynthesis-related enzymes from Ralstonia eutropha, including PHA synthase (PhaB), β-ketothiolase (PhaA) and reductase (PhaB), and a DNA fragment containing parB(hok/sok) locus were obtained by digestion of pSYL105 (see: Lee et al., Biotechnol. Bioeng., 44: 1337-1347, 1994) with restriction endonuclease XbaI, which were then introduced into XbaI site of pACYC184 (New England Biolabs, USA) capable of coexisting with plasmids derived from pUC19, finally to construct p5184.

Further, to prepare a microorganism harboring a gene encoding PHA biosynthesis-related enzymes in an integrated form with its own chromosome, a gene encoding PHA biosynthesis-related enzymes from Ralstonia eutropha, which was obtained by digestion of pSYL105 (see: Lee et al., Biotechnol. Bioeng., 44:1337-1347, 1994) with restriction endonuclease BamHI, was introduced within phosphotransacetylase (Pta) locus in the chromosome of E. coli B (ATCC 11303) by means of homologous recombination, and prepared E. coli B-PHA+, a mutant E. coli for PHA biosynthesis.

E. coli XL1-Blue (Stratagene Cloning System, USA) was transformed with the said pUC19Red and p5184 through electroporation, to prepare a recombinant E. coli XL1-Blue/pUC19Red;p5184, which contains a gene encoding PHA biosynthesis-related enzymes and a gene encoding intracellular PHA depolymerase. Then, by transforming the said E. coli B-PHA+ with said pUC19Red_stb through electroporation, a recombinant E. coli B-PHA+/pUC19Red_stb having both a gene encoding PHA biosynthesis-related enzymes and a gene encoding intracellular PHA depolymerase was prepared. Each of the recombinant microorganisms was cultured in media containing proper carbon source, then the concentration of (R)-hydroxybutyrate produced therefrom was measured, respectively. As a result, it was found that (R)-hydroxybutyrate can be produced efficiently by culturing each of the said recombinant microorganisms, and appropriate conditions for their culture was established. For the production of (R)-3-hydroxybutyrate, a recombinant microorganism co-transformed with both a gene encoding intracellular PHA depolymerase from Ralstonia eutropha and a gene encoding PHA biosynthesis-related enzymes from Alcaligenes latus or Ralstonia eutropha, is preferably cultured for 30 to 70 hours.

Although the present invention employed two plasmid vectors of pUC19 with pACYC184 that can coexist with each other, it is obvious for those skilled in the art that any combination of the vectors that can coexist would be applicable in the invention. For the genes encoding PHA biosynthesis-related enzymes and intracellular PHA depolymerase, it is obvious for those skilled in the art that genes for the same role from any microorganism other than Ralstonia eutropha is similarly applicable if the gene can be active in host microorganism. In addition, it is obvious for those skilled in the art that for chromosomal integration of a gene encoding PHA biosynthesis-related enzymes, it can be incorporated within any locus other than phosphotransacetylase locus, as far as it does not have a critical influence on the metabolism of the microorganism. Moreover, it is obvious for those skilled in the art that host microorganisms for the expression of the plasmids can be any E. coli strain other than E. coli XL1-Blue, and that for chromosomal integration of a gene encoding PHA biosynthesis-related enzymes, any E. coli strain other than E. coli B may be applicable if it is able to have an altered chromosome. Further, it is obvious for those skilled in the art that a host microorganism of the present invention can be any microorganism being capable of expressing the said enzymes other than E. coli.

The present invention is further illustrated in the following examples, which should not be taken to limit the scope of the invention.

### EXAMPLE 1

### Cloning of a Gene Encoding Intracellular PHA Depolymerase from Ralstonia eutropha

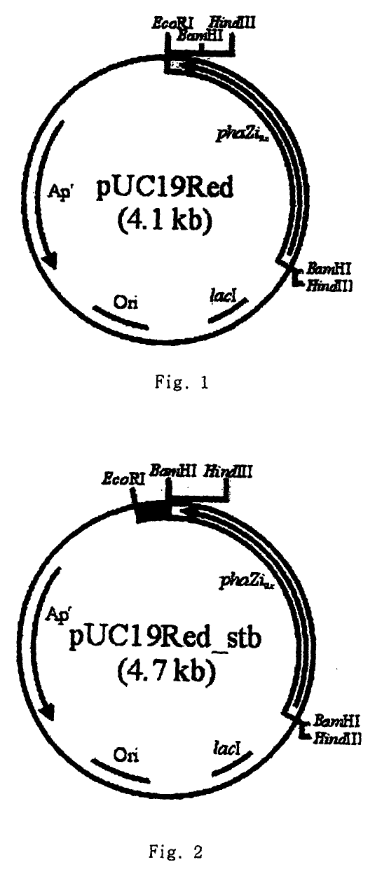

For the cloning of intracellular PHA depolymerase from Ralstonia eutropha, PCR was performed by using a chromosomal DNA isolated from Ralstonia eutropha according to Marmur et al's method (see: Marmur, J. Mol. Biol., 3:208-218, 1961) as a template and using primer 1,5′-GCTCTAGAGGATCCTTGTTTTCCGCAGCAACAGCT-3′ (SEQ. ID No: 4)) and primer 2,5′-CGGGATCCAAGCTTACCTGGTGGCCGAGGC-3′ (SEQ. ID No: 5), complementary to the sequence of a gene encoding intracellular PHA depolymerase from Ralstonia eutropha (see: Saito and Saegusa, GenBank Sequence Database, AB017612, 2001). In carrying out the PCR, pre-denaturation was first made at 95° C. for 5 min and then, 30 cycles of reactions were performed, each of which comprises the steps of: denaturation at 95° C. for 50 sec; annealing at 55° C. for 1 min 10 sec; and, elongation at 72° C. for 3 min. Finally, post-elongation was made at 72° C. for 7 min. The resulting DNA fragment was digested with a restriction endonuclease BamHI, and 1.4 kbp of DNA fragment was fractionated on agarose gel electrophoresis, and ligated into pUC19 (see: Sambrook et al., Molecular Cloning, A Laboratory Manual 2nd Ed., Cold Spring Harbor Laboratory Press, Cold Spring Harbor, N.Y., 1989; New England Biolabs, USA) digested with the same restriction endonuclease, to construct pUCRed. Then, E. coli XL1-Blue was transformed with pUC19Red by the technique of electroporation and then, was screened for successful transformants on LB plate media (Yeast extract, 5 g/L; tryptone, 10 g/L; NaCl, 10 g/L) containing ampicillin (50 mg/L) and bacto-agar (15 g/L), finally to prepare a recombinant microorganism of XL1-Blue/pUC19Red. Comparison with the sequences accessible on GenBank™ revealed that sequence of the cloned DNA fragment is a gene encoding intracellular PHA depolymerase from Ralstonia eutropha including its innate promoter region for constitutive expression and its ribosome-binding site. FIG. 1 is a genetic map of pUC19Red, a recombinant plasmid of the present invention.

Then, the digestion of pSYL105 with restriction endonuclease EcoRI and BamHI was performed to obtain a DNA fragment including parB(hok/sok) locus (see: Gedes, Bio/Technology, 6:1402-1405, 1988), which was then introduced into pUC19Red to construct pUC19Red_stb. FIG. 2 is a genetic map of pUC19Red_stb, a recombinant plsmid of the present invention.

### EXAMPLE 2

### Construction of a Plasmid Containing a Gene Encoding PHA Biosynthesis-Related Enzymes from Ralstonia eutropha, which can Coexist with Other Plasmids in E. coli

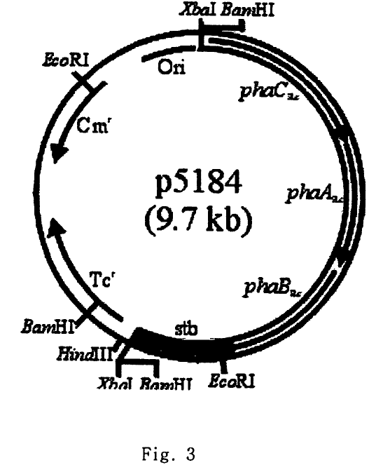

Since pUC19Red and pUC19Red_stb prepared in Example 1 can express only those enzymes related to depolymerization of intracellular PHAs into monomers, E. coli should be transformed with another plasmid containing a gene encoding PHA biosynthesis-related enzymes to prepare a recombinant microorganism producing (R)-3-hydroxybutyrate. To this end, a plasmid p5184 containing a gene encoding PHA biosynthesis-related enzymes from Ralstonia eutropha and being capable of coexisting with other plasmids derived from pUC19 containing ColE1-compatible origin of replication, was prepared as follows: First, pSYL105 was digested with restriction endonuclease XbaI, then subject to agarose gel electrophoresis to obtain 5.6 kbp of DNA fragment containing a gene encoding PHA biosynthesis-related enzymes from Ralstonia eutropha and hok/sok gene. Then, the DNA fragment was incorporated into pACYC184 (New England Biolabs, USA) having a p15A origin of replication, which was digested with the same restriction endonuclease, finally to prepare p5184. FIG. 3 is a genetic map of p5184, a recombinant plasmid of the present invention.

### EXAMPLE 3

### Preparation of E. coli Harboring a Gene Encoding PHA Biosynthesis-Related Enzymes in an Integrated Form with its Chromosome

To insert a gene encoding PHA biosynthesis-related enzymes within E. coli phosphotransacetylase (Pta) locus by way of homologous recombination (see: Yu et al., Proc. Natl. Acad. Sci., USA, 97:5978-5983, 2000), PCR was carried out for the amplification of a gene encoding phosphotransacetylase with two halves of fragments, by using a chromosomal DNA of E. coli B (ATCC 11303) as a template and using primer 3,5′-GCGAATTCTTTAAAGACGCGCGCATTTCTAAACT-3′ (SEQ ID No: 6), primer 4, 5′-GCGGTACCGAGCTCCGGGTTGATCGCACAGTCA-3′ (SEQ ID No: 7), primer 5,5′-GGCGAGCTCGCGCATGCCCGACCGCTGAACAGCTG-3′ (SEQ ID No: 8) and primer 6,5′-GCAAGCTTTTTAAAGCGCAGTTAAGCAAGATAATC-3′ (SEQ ID No: 9). And, one of the half fragment was digested with restriction endonuclease EcoRI and KpnI while the other half fragment was digested with SphI and HindIII, and then were ligated into a pUC19 plasmid digested with the same restriction endonucleases, respectively. A kanamycin resistance gene was amplified by PCR using pACYC177(New England Biolabs, USA) as a template and using primer 7,5′-GCTCTAGAGAGCTCAAAGCCACGTTGTGTCTCAAA-3′ (SEQ ID No: 10) and primer 8,5′-GCGCATGCTTAGAAAAACTCATCGAGCATC-3′ (SEQ ID No: 11), and the resulting fragment was digested with SalI and SphI and then ligated into a plasmid containing the said two fragments of pta gene. A gene fragment including a gene encoding PHA biosynthesis-related enzymes, which was prepared from the digestion of pSYL105 with restriction endonuclease BamHI, was also ligated into BamHI site of the plasmid. Then, the plasmid was digested with restriction endonuclease DraI and then the resulting fragments including all the said genes were fractionated on agarose gel electrophoresis.

E. coli B (ATCC 11303) transformed with pTrcEBG (see: Korean Patent Appln. No. 10-2001-48881), was induced by IPTG to be used for competent cell, which was then transformed with the said fragments by electroporation, and then, the colonies whose chromosomes were integrated with the said fragments were selected on Luria-Bertani (LB) plate media containing kanamycin. Then, colonies lacking pTrcEBG were selected as a result of subculture in LB liquid media containing kanamycin, and PCR amplification of the gene encoding PHA biosynthesis-related enzymes using chromosomal DNA as a template, was followed to confirm the correct integration of the gene into chromosome. PHA synthesis through the culturing them in media containing glucose, made sure that PHA biosynthesis-related enzymes have a biological activity. Then, the selected recombinant E. coli was named as ‘E. coli B-PHA+’.

### EXAMPLE 4

### Preparation of a Recombinant Microoranism Producing (R)-hydroxycarboxylic Acids

E. coli XL1-Blue was transformed with both pUC19Red and p5184 prepared in Examples 1 and 2 by way of electroporation to prepare recombinant E. coli XL1-Blue/pUC19Red;p5184. And, E. coli B-PHA+ prepared in Example 3 was transformed with pUC19Red_stb prepared in Example 1 by electroporation to prepare recombinant E. coli B-PHA+/pUC19Red_stb.

### EXAMPLE 5

### Preparation of (R)-hydroxybutyrate

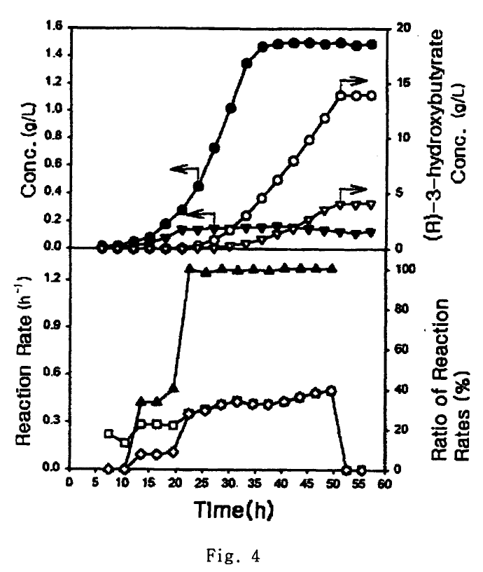

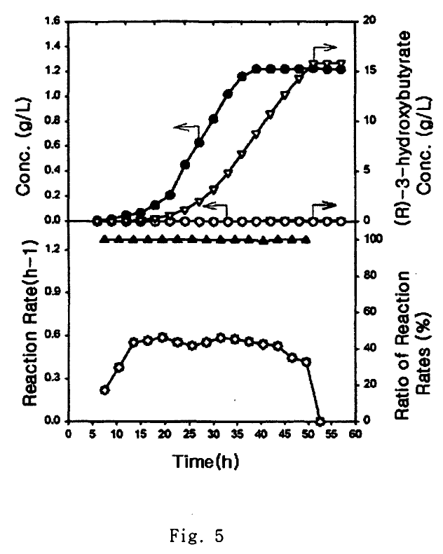

Recombinant E. coli XL1-Blue/pSYL105Red (see: Korean Patent Appln. No. 10-2000-002615) was cultured in LB media containing 50 mg/L ampicillin for 12 hours, and then 1 in Q of the culture was inoculated in 250 ml flask containing 100 ml of R media with 20 g/L of glucose, 20 mg/L of thiamin and 50 mg/L of ampicillin (see: Choi et al., Appl. Environ. Microbiol., 64:4897-4903, 1998), and then cultured at 37° C. in a shaking incubator at 250 rpm. Then, the recombinant E. coli XL1-Blue/pUC19Red;p5184 was cultured for 12 hours in LB media containing 50 mg/L of ampicillin and 50 mg/L of chlorampenicol and then 1 ml of the culture was inoculated in 250 ml flask containing 100 ml of R media with 20 g/L of glucose, 20 mg/L of thiamin, 50 mg/L of ampicillin and 50 mg/L of chloramphenicol (see: Choi et al., Appl. Environ. Microbiol., 64:4897-4903, 1998), and was cultured at 37° C. in a shaking incubator at 250 rpm. FIG. 4 is a graph showing the concentrations of dried-microorganism (●), polyhydroxybutyrate (PHB) (▾) and PHB monomers prepared before (∇) and after () pyrolysis under a basic condition, the rates of PHA biosynthesis ( [symbol] ) and PHB degradation (⋄), and the ratio of PHB degradation rate/biosynthesis rate (▴), depending on the elapsed time during the culture of recombinant E. coli XL1-Blue/pSYL105Red. FIG. 5 is a graph showing the concentrations of dried-microorganism (●), PHB (▾) and PHB monomers prepared before (∇) and after () pyrolysis under a basic condition, and the rates of PHA biosynthesis ( [symbol] ) and PHB degradation (⋄), and the ratio of PHB degradation rate/biosynthesis rate (▴), depending on the elapsed time during the flask culture of recombinant E. coli XL1-Blue/pUC19Red;p5184. As can be seen in the results of FIGS. 4 and 5, it was demonstrated that: recombinant E. coli XL1-Blue/pUC19Red;p5184 showed rather rapid PHA biosynthesis than expectation that expression level of the PHA biosynthesis-related enzymes is lowered due to relatively low copy number of its plasmid; and, the rate of PHA degradation became high. If PHA is degradated into a form smaller than dimeric form, it may be released out of a microorganism without difficulty. Therefore, (R)-hydroxybutyrate was prepared mostly in a dimeric form, which was, in turn, easily changed into a monomeric form by pyrolysis under a basic condition (see: Lee et al., Biotechnol. Bioeng., 65:363-368, 1999; Korean Patent No. 250830; PCT International Appln. No. PCT/KR98/00395).

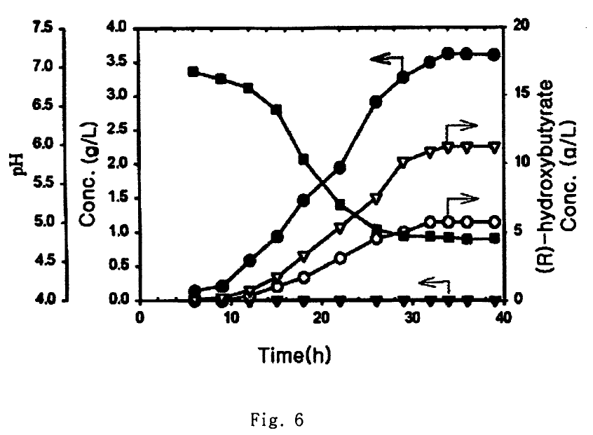

In addition, the recombinant E. coli B-PHA+/pUC19Red_stb prepared in Example 4 was cultured in a batch-wise in 2.5 L jar fermentor (KoBiotech, Korea) containing 1.5 L of R media (initial pH 7.0) with 20 g/L of glucose (see: Choi et al., Appl. Environ. Microbiol., 64:4897-4903, 1998), at 37° C. in a shaking incubator at 500 rpm while not adjusting pH during the culture. FIG. 6 is a graph showing the concentrations of dried-microorganism (●), PHB (▾) and PHB monomers prepared before (∇) or after () pyrolysis under a basic condition and pH (▮) depending on the elapsed time during the batch culture of a recombinant E. coli B-PHA+/pUC19Red_stb of the present invention. As can be seen in FIG. 6, 34 hours of culture allowed the total production of 11.8 g/L (R)-3-hydroxybutyrate and its dimers, which is estimated to be a final yield of up to 59%. Further, it was examined that the plasmids were stable enough since no microorganism lacking its plasmid was found during the culture, indicating that there is no need to use antibiotics for maintaining its plasmid.

### EXAMPLE 6

### Simultaneous Production of (R)-hydroxybutyrate and (R)-hydroxyvalerate

The recombinant E. coli XL1-Blue/pUC19Red_stb was cultured for 12 hours in LB media, and 1 ml of the culture was inoculated in 250 ml flask containing 100 ml of R media with 10 g/L of glucose and 1 g/L of propionic acid 20 mg/L (see: Choi et al., Appl. Environ. Microbiol., 64:4897-4903, 1998), and then cultured at 37° C. in a shaking incubator at 250 rpm. After 48 hours of culture, pyrolysis was performed under a basic condition, and HPLC analysis of the resultant revealed that 4.1 g/L of (R)-3-hydroxybutyrate and 0.4 g/L of (R)-3-hydroxybutyrate were prepared, respectively.

As clearly illustrated and demonstrated as above, the present invention provides recombinant microorganisms carrying both a gene encoding intracellular PHA depolymerase and a gene encoding polyhydroxyalkanoate (PHA) biosynthesis-related enzymes, and a process for preparing optically pure (R)-hydroxycarboxylic acids by culturing the said recombinant microorganisms where biosynthesis and degradation of PHA occur simultaneously. In accordance with the present invention, (R)-hydroxycarboxylic acids can be released from the recombinant microorganisms into culture media after depolymerizing most PHA produced from the said microorganisms, which makes possible practical preparation of (R)-hydroxycarboxylic acids in a simple manner with reduced waste of substrates to increase the productivity, finally to allow the mass production of various optically pure (R)-hydroxycarboxylic acids.

While the present invention has been shown and described with reference to the particular embodiments, it will be apparent to those skilled in the art that many changes and modifications may be made without departing from the spirit and scope of the invention as defined in the claims.

1. A recombinant microorganism producing optically pure (R)-hydroxycarboxylic acid, which is co-transformed with a recombinant plasmid containing a gene encoding intracellular polyhydroxyalkanoate (PHA) depolymerase (SEQ ID No: 1) and a recombinant plasmid containing a gene encoding PHA biosynthesis-related enzymes.

2. The recombinant microorganism of claim 1, wherein the gene encoding intracellular PHA depolymerase and the gene encoding PHA biosynthesis-related enzymes are derived from Ralstonia eutropha.

3. The recombinant microorganism of claim 1, wherein the copy number of the gene encoding PHA depolymerase is higher than that of the gene encoding PHA biosynthesis-related enzymes.

4. The recombinant microorganism of claim 1, wherein the microorganism is Escherichia coli.

5. The recombinant microorganism of claim 1, wherein the microorganism is E. coli XL 1-Blue.

6. The recombinant microorganism of claim 1, wherein (R)-hydroxycarboxylic acid is (R)-3-hydroxybutyrate or (R)-3-hydroxyvalerate.

7. The recombinant microorganism of claim 6, wherein (R)-3-hydroxybutyrate and (R)-3-hydroxyvalerate is in monomeric or dimeric form.

8. Escherichia coli XLI-Blue/pUC19Red;p5184 producing optically pure (R)-hydroxycarboxylic acid.

9. A recombinant microorganism producing optically pure (R)-hydroxycarboxylic acid, which is transformed with a recombinant DNA encoding intracellular PHA depolymerase while containing a gene encoding PHA synthesis-related enzymes in an integrated form with its chromosome.

10. The recombinant microorganism of claim 9, wherein the gene encoding PHA synthesis-related enzymes is present within phosphotransacetylase (Pta) locus in an integrated form with its chromosome.

11. The recombinant microorganism of claim 9, wherein the gene encoding intracellular PHA deploymerase is present in a plasmid in an inserted form.

12. The recombinant microorganism of claim 9, wherein the gene encoding intracellular PHA depolymerase is present in a plasmid together with parB(hok/sok) locus (SEQ ID No: 10) derived from E. coli R1 in an inserted form.

13. The recombinant microorganism of claim 9, wherein the gene encoding intracellular PHA depolymerase and the gene encoding PHA biosynthesis-related enzymes are derived from Ralstonia eutropha.

14. The recombinant microorganism of claim 9, wherein the microorganism is Escherichia coli.

15. The recombinant microorganism of claim 9, wherein the microorganism is E. coli XL1-Blue or E. coli B.

16. The recombinant microorganism of claim 9, wherein (R)-hydroxycarboxylic acid is (R)-3-hydroxybutyrate or (R)-3-hydroxyvalerate.

17. The recombinant microorganism of claim 16, wherein (R)-3-hydroxybutyrate and (R)-3-hydroxyvalerate are monomeric or dimeric form.

18. A recombinant microorganism, E. coli B-PHA+/pUC19Red_stb producing optically pure (R)-hydroxycarboxylic acid.

19. A process for preparing optically pure (R)-hydroxycarboxylic acid, which comprises the steps of culturing the recombinant microorganism of claim 1 and obtaining (R)-hydroxycarboxylic acid from the culture.

20. The process of claim 19, wherein the culturing is carried out by continuous or batch process.

21. The process of claim 19, wherein (R)-hydroxycarboxylic acid is (R)-3-hydroxybutyrate or (R)-3-hydroxyvalerate.

22. The process of claim 21, wherein (R)-3-hydroxybutyrate and (R)-3-hydroxyvalerate are monomeric or dimeric form.

23. A process for preparing optically pure (R)-hydroxycarboxylic acid, which comprises the steps of culturing the recombinant microorganism of claim 9 and obtaining (R)-hydroxycarboxylic acid from the culture.

24. The process of claim 23, wherein the culturing is carried out by continuous or batch process.

25. The process of claim 23, wherein (R)-hydroxycarboxylic acid is (R)-3-hydroxybutyrate or (R)-3-hydroxyvalerate.

26. The process of claim 25, wherein (R)-3-hydroxybutyrate and (R)-3-hydroxyvalerate are monomeric or dimeric form.

27. A process for preparing optically pure (R)-hydroxycarboxylic acid, which comprises the steps of culturing E. coli XL1-Blue/pUC19Red;p5184 and obtaining (R)-hydroxycarboxylic acid from the culture.

28. A process for preparing optically pure (R)-hydroxycarboxylic acid, which comprises the steps of culturing E. coli B-PHA+/pUC19Red_stb and obtaining (R)-hydroxycarboxylic acid from the culture.
